(19) United States
(12) Patent Application Publication
(10) Pub. No.: US 2025/0359913 A1
(43) Pub. Date: Nov. 27, 2025

(54) Implant Removal Instrument

(71) Applicant: Howmedica Osteonics Corp., Mahwah, NJ (US)

(72) Inventors: Shammodip Roy, Wayne, NJ (US); Sharon Gaukler, Riverdale, NJ (US); Peter Tulkis, Paramus, NJ (US)

(21) Appl. No.: 19/214,230

(22) Filed: May 21, 2025

Related U.S. Application Data
(60) Provisional application No. 63/651,182, filed on May 23, 2024.

(51) Int. Cl. A61B 17/92; A61F 2/46

(52) U.S. Cl. CPC A61B 17/92; A61B 2017/922; A61F 2002/4619

(57) ABSTRACT

An implant extraction instrument includes a shaft, a first gripping member and a second gripping member. The first gripping member is movable relative to the shaft and includes an elongate portion and a protrusion extending from the elongate portion in a direction transverse to a central longitudinal axis of the shaft. The second gripping member is connected to the first gripping member, a position of the second gripping member being adjustable relative to the first gripping member. When the protrusion and the second gripping member are positioned to grip a first implant component therebetween, a position of the shaft relative to the first gripping member is controllable to cause the first implant component to be pulled away from a second implant component to which the first implant component is attached.

### CROSS-REFERENCE TO RELATED APPLICATION

The present application claims the benefit of the filing date of U.S. Provisional Patent Application No. 63/651,182, filed May 23, 2024, the disclosure of which is hereby incorporated by reference herein in its entirety.

### BACKGROUND

A wide range of clinical indications for arthroplasty and trauma procedures involve intra-operative removal of a joint implant such as a hip joint implant. Depending on the age of the in vivo implant, patient indications, anatomy and type of implant, such implant removal step may be extremely time consuming and difficult, and may impact the overall duration of surgery as well as contribute to increased risk of surgical complications. When the implant is a hip implant, removal may involve extraction of a cementless acetabular liner from a well-fixed acetabular shell or a femoral head from a well-fixed stem. Both of these constructs are typically taper-locked, and it is important for the removal technique to be able to break the taper lock of the implant component to be extracted both expeditiously and in a manner that is non-destructive to the well-fixed implant.

Existing extraction techniques include use of a mallet to impact an implant component with sufficient energy to dissociate the locked tapers in the implant. However, such manual techniques are subject to variability in application of forces and may require an onerous amount of time and effort. Other existing techniques include the use of powered tools to perform a similar function. However, the use of powered tools may cause undesirable forces to be applied through reciprocating movement in the tool, e.g., forces into the implant that may cause a bone fracture. Further, powered tools may vibrate in a manner such that control of the tool is limited.

Accordingly, a need exists for improved instrumentation for the extraction of implant components from an in vivo implant.

### BRIEF SUMMARY

In a first aspect, the present disclosure relates to an implant extraction instrument. In a first example of a first embodiment, the implant extraction instrument includes a shaft, a first gripping member movable relative to the shaft and a second gripping member connected to the first gripping member. The first gripping member including an elongate portion and a protrusion extending from the elongate portion in a direction transverse to a central longitudinal axis of the shaft. A position of the second gripping member is adjustable relative to the first gripping member. When the protrusion and the second gripping member are positioned to grip a first implant component therebetween, a position of the shaft relative to the first gripping member is controllable to cause the first implant component to be pulled away from a second implant component to which the first implant component is attached.

In a second example of the first embodiment, the first gripping member may be translatable relative to the shaft. In a third example, a rotational position of the first gripping member relative to the shaft may be fixed. In a fourth example, the shaft may be an outer shaft and the first gripping member may be a clamp, and the outer shaft and the clamp may be operatively connected through a biasing member. In a fifth example, a force pushing the outer shaft toward the clamp may be attenuated by the biasing member. In a sixth example, the second gripping member may include a locking member to set a position of an implant-interfacing surface of the second gripping member relative to the first gripping member. In a seventh example, the implant extraction instrument may include a housing connected to the shaft, the housing including a motor operatively connected to the shaft. In an eighth example, the first gripping member may be disposed within a cannulation of the shaft and the first gripping member may include a proximal shaft and a distal shaft configured such that rotation of the proximal shaft causes the distal shaft to translate relative to the proximal shaft. In a ninth example, the second gripping member may be pivotably attached to the first gripping member.

In a first example of a second embodiment, an implant extraction instrument may include a handle member, a grip member movably attached to the handle member and a biasing member with a first end connected to the handle member and a second end connected to the grip member. The handle member includes a shaft and extends from a proximal end to a distal end. The grip member is movably attached to the shaft proximate the distal end of the shaft and includes a gripping arm configured to hold an implant. The biasing member includes a first end connected to the shaft and a second end connected to the grip member. When the grip member is attached to an implant in a patient, the shaft is movable relative to the grip member such that when the shaft is pushed toward the grip member, at least a portion of a force in the shaft that is associated with the pushing is absorbed by the biasing member, and when the shaft is pulled away from the grip member, a force in the shaft that is associated with the pulling is transferred to the grip member.

In a second example of the second embodiment, the shaft may include a slot and the grip member may include a pin disposed through the slot, a position of the pin within the slot changing with movement of the grip member relative to the shaft. In a third example, when the pin abuts a distal end of the slot, the proximal end of the handle member and the pin may be at a maximum distance with respect to each other. In a fourth example, a portion of the grip member may be disposed within the shaft and the biasing member may be entirely disposed within the shaft. In a fifth example, the gripping arm may have a free end with a protrusion extending transversely relative to the gripping arm, the gripping arm being configured to hold the implant. In a sixth example, the protrusion of the gripping arm may be offset from a central longitudinal axis of the shaft. In a seventh example, the gripping arm may be parallel to the shaft. In an eighth example, the implant extraction instrument may include a supplemental grip member movably attached to the grip member, the supplemental grip member being shaped to clamp the implant in a direction different from that of the gripping arm against the implant. In a ninth example, the implant extraction instrument may include a locking member configured to fix a position of the supplemental grip member relative to the grip member. In a tenth example, the gripping arm may have a first engagement portion and the supplemental grip member may have a second engagement portion, the second engagement portion being a softer material than the first engagement portion.

In a first example of a third embodiment, the implant extraction instrument includes an outer shaft, an inner shaft movably disposed in the outer shaft, and a clamp arm rotatably attached to the outer shaft. The outer shaft has an elongate dimension extending along a central longitudinal axis. The inner shaft includes a proximal portion and a distal portion translatable relative to the proximal portion, the distal portion including a tip protrusion extending transversely relative to the central longitudinal axis. Further, the clamp arm includes an end portion moveable relative to the tip protrusion. The tip protrusion and the end portion are configured to be movable to grip an object therebetween.

In a second example of the third embodiment, the outer shaft may include an elongate prong extending from a distal end of the outer shaft, the elongate prong extending distally of a lumen of the outer shaft such that an end of the distal portion of the inner shaft is positionable adjacent to the elongate prong outside of the lumen. In a third example, the outer shaft may include two elongate prongs extending from a distal end of the outer shaft, the two elongate prongs being disposed radially outward of a path of axial translation of the inner shaft such that the inner shaft is positionable between the two elongate prongs. In a fourth example, the proximal portion of the inner shaft may be rotatable to cause the distal portion of the inner shaft to translate relative to the proximal portion, thereby translating tip protrusion relative to a distal end of the outer shaft. In a fifth example, the proximal portion may include a threaded end portion and the distal portion may include a threaded receiving cavity, the threaded end portion being threadably received in the threaded receiving cavity. In a sixth example, a rotational position of the distal portion relative to the outer shaft may be fixed. In a seventh example, the clamp arm may be pivotable relative to the outer shaft. In an eighth example, a pivot axis of the clamp arm relative to the outer shaft may be at a first location on the outer shaft and a locking member of the clamp arm configured to lock a rotational position of the clamp arm relative to the outer shaft may be at a second location on the outer shaft spaced apart from the first location.

In a second aspect, the present disclosure relates to a method of removing a first implant component from a second implant component using an extraction instrument. In a first example of a first embodiment, a method of removing a first implant component from a second implant component includes: positioning a gripping end of the extraction instrument under a surface of an engagement region of the first implant component such that at least a portion of the first implant component is closer to a proximal shaft of the extraction instrument than the gripping end, the gripping end being distal to the proximal shaft; and causing activation of a reciprocating movement of the proximal shaft relative to the gripping end such that force in the proximal shaft is transferred to the gripping end and the gripping end pulls on the engagement region of the first implant component relative to the second implant component to which the first implant component is attached.

In a second example of the first embodiment, when the proximal shaft is in a pushing phase of the reciprocating movement, the proximal shaft moves closer to the gripping end attenuating force transferred from the proximal shaft to the gripping end. In a third example, when the shaft is in a pushing phase of the reciprocating movement, a biasing member mounted distally to the proximal shaft attenuates a force against the biasing member to reduce force transferred to the gripping end. In a fourth example, the method may include adjusting a supplemental gripping member to clamp onto a second surface of the first implant component. In a fifth example, the method may include tightening a locking member onto the supplemental gripping member to clamp the supplemental gripping member onto the second surface of the first implant component. In a sixth example, activation of the reciprocating movement may include pressing a button to cause a motor to activate, the motor being operatively connected to the proximal shaft. In a seventh example, the second implant component may be anchored within a bone of a patient prior to commencing the method. In an eighth example, the first implant component may be an acetabular liner and the second implant component may be an acetabular shell.

### BRIEF DESCRIPTION OF THE DRAWINGS

A more complete appreciation of the subject matter of the present disclosure and of the various advantages thereof can be realized by reference to the following detailed description in which reference is made to the accompanying drawings in which:

FIG. 1 is a perspective view of an extraction instrument according to one embodiment of the present disclosure;

FIG. 2 is a partial side view of the extraction instrument of FIG. 1;

FIGS. 3-4 are partial side views of the extraction instrument of FIG. 1 with moving parts in respective first and second reciprocating motion positions;

FIG. 5 is a perspective view of an extraction instrument according to one embodiment of the present disclosure;

FIG. 6 is a partial perspective view of the extraction instrument of FIG. 5;

FIGS. 7 and 8 are perspective and cross-sectional views, respectively, of an extraction instrument according to one embodiment of the present disclosure;

FIG. 9 is a front view of an extraction instrument according to one embodiment of the present disclosure;

FIG. 10 is a side view of the extraction instrument of FIG. 9;

FIG. 11 is a partial perspective view of the extraction instrument of FIG. 9;

FIG. 12 is a partial side view of the extraction instrument of FIG. 9;

FIGS. 13A-B, 14A-B, 15A-B and 16A-B illustrate steps in a method of using the extraction instrument of FIG. 9 according to one embodiment of the present disclosure;

FIG. 17 is a perspective view of a shaft of an extraction instrument according to one embodiment of the present disclosure;

FIG. 18 is a perspective view of an extraction instrument according to one embodiment of the present disclosure and

FIGS. 19-21 illustrate steps in a method of using the extraction instrument of FIG. 18 according to one embodiment of the present disclosure.

### DETAILED DESCRIPTION

As used herein, the term “distal” refers to that portion of an instrument or component thereof which is farther from the user while the term “proximal” refers to that portion of the instrument or component thereof which is closer to the user. The term “medial” means closer to or toward the midline of the body, and the term “lateral” means further from or away from the midline of the body. The term “inferior” means closer to or toward the feet, and the term “superior” means closer to or toward the crown of the head. As used herein, the terms “about,” “approximately,” “generally,” and “substantially” are intended to mean that slight deviations from absolute are included within the scope of the term so modified.

In one aspect, the present disclosure relates to an implant extraction instrument. In some examples, and as shown in the depicted embodiments, the implant extraction instrument, also referred to as an extraction instrument, may be used to remove a liner from a shell of an acetabular implant or to remove a femoral head from a femoral stem. In other examples, the extraction instrument may be used to separate components of a glenoid implant or to separate components of segmental limb salvage implants. It should be appreciated that such uses of the extraction instrument are not limiting and the instrument may be employed for other uses. For example, the extraction instrument may be used to pry apart other two-part surgical implants. In some of these examples, the extraction instrument may be used to separate implant components having one or more taper locked junctions between them.

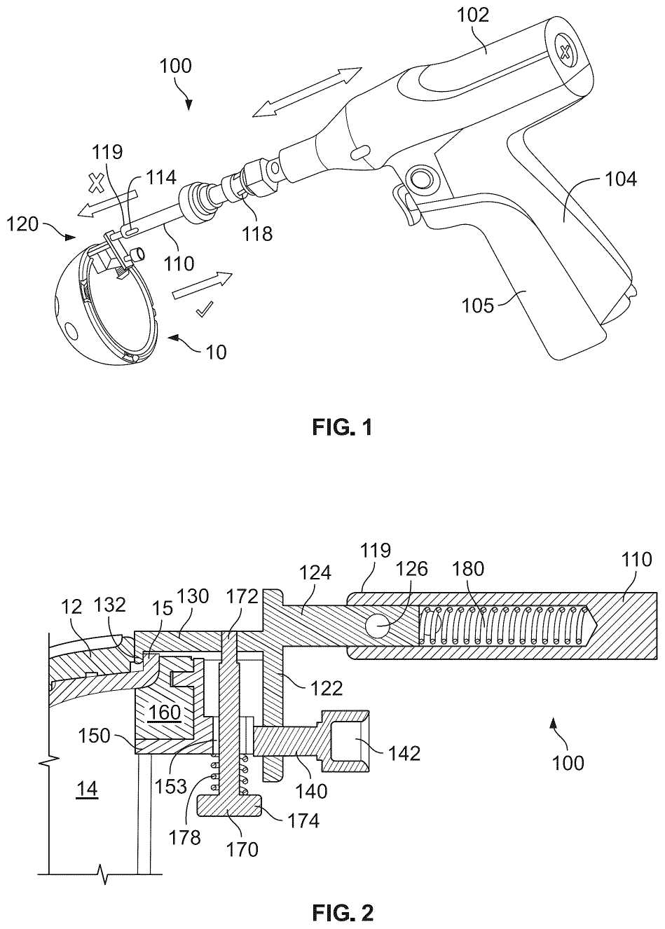

In one embodiment, an extraction instrument is identified by reference numeral 100 and is shown in FIGS. 1-4. Extraction instrument 100 includes a housing 102, a shaft 110 attached to housing 102, and a clamp 120 attached to shaft 110.

Housing 102 includes a pistol-grip handle 104 and a trigger 105 operatively connected thereto. Built into housing 102 is a mechanism to control reciprocating action of a component either internal to housing 102 or on an exterior of the housing and movable relative to a remainder of the housing, where such component is directly attached to shaft 110. In one specific example, the mechanism may be a linear motor. In another specific example, the mechanism may be a brushless DC motor connected via a bevel connection to a rotating pawl that impacts and pushes a floating drive shaft. In some examples, operation of such mechanism may be controlled through the supply of power to the mechanism. In the depicted embodiment, depression of trigger 105 activates the mechanism to thereby cause shaft 110, operatively connected to the mechanism, to reciprocate along its longitudinal axis and relative to housing 102. In variations, the housing part of the extraction instrument may be a structure other than a pistol-grip handle arrangement. For example, the housing may be an elongate housing with a button to control activation of the actuation mechanism.

Shaft 110 extends from a first end 118 attached to housing 102 to a second end 119 opposite the first end, the second end attached to clamp 120. Proximate second end 119, shaft 110 includes an internal cavity 112 with an elongate dimension aligned with the central longitudinal axis of shaft 110, as shown in FIGS. 2-4. A biasing member, e.g., first spring 180 in the depicted embodiment, is disposed at an internal end of cavity 112, and a portion of clamp 120 is received on an opposite end of first spring 180. In some examples, a biasing member other than a spring may be included. Also proximate second end 119 of shaft 110 are slots 114 (one shown in FIGS. 1 and 2-4) that are disposed opposite each other and extend through a wall of shaft 110 so that cavity 112 is in communication with an external surface of shaft 110 in a radial direction. Such slots 114 receive a pin 126 of clamp 120 or another connection element to prevent clamp 120 from separating from shaft 110, while also allowing shaft 110 to move relative to clamp 120. Slots 114 are enclosed along a radial sidewall of shaft 110 such that an entirety of the slot is spaced apart from second end 119 of shaft 110. In some examples, the shaft may include only a single slot on one side, with a first end of the pin extending into the slot, while a second end opposite the first end does not protrude from the receiving post, allowing the receiving post to reciprocate within the shaft.

Clamp 120 includes a dual-clamping feature to grip an object such as an implant component. In the depicted embodiment, clamp 120 is shown gripping a liner 14 of an acetabular implant 10. As shown in FIGS. 2-4, clamp 120 includes a base 122, a receiving post 124 extending from base 122 in a proximal direction, a gripping arm 130 extending from base 122 in a distal direction, and an optional retention plate 138 also extending from base 122 in the distal direction, but spaced apart from gripping arm 130. Receiving post 124 extends from base 122 to a free end and includes a transverse opening therethrough that receives pin 126. Pin 126 is longer than a diameter of the receiving post. A portion of receiving post 124 is disposed in cavity 112 of shaft 110, and pin 126 holds receiving post 124 relative to shaft 110 along a range of relative positions limited by a size of slot 114 in shaft 110. Pin 126 is attached in a set position relative to receiving post 124 when disposed therein. Attachment may be through an adhesive or other securement means. It is also contemplated that in some examples, the pin and receiving post combination may be formed monolithically. In still further examples, the pin may be substituted with a protrusion, optionally monolithic with the receiving post, that is configured to fit within the slot but that has a shape that is otherwise different from the pin. When assembled, a central longitudinal axes of receiving post 124 and shaft 110 may be coincident. Further, while a shape of receiving post 124 and shaft 110 may be generally cylindrical as shown, other shapes are contemplated, such as cross-sectional shapes that are oblong or polygonal, with the shapes of the receiving post and the shaft being complementary.

With continued reference to clamp 120, gripping arm 130 extends from base 122 in a direction opposite that of receiving post 124. In the depicted embodiment, gripping arm 130 is parallel to and offset from an elongate dimension of receiving post 124. In other examples, the gripping arm and the receiving post may extend along the same axis. In still further examples, an elongate dimension of the gripping arm may be at an angle relative to the elongate dimension of the receiving post. Gripping arm 130 extends to a free end remote from base 122. A protrusion 132 extends from a surface of the gripping arm proximate the free end. Such protrusion 132 extends at an angle relative to the elongate dimension of gripping arm 130. In some examples, and as shown in FIGS. 2-4, protrusion 132 extends orthogonally relative to the elongate dimension of the gripping arm. Protrusion 132 is configured to provide one means of gripping or otherwise clamping onto an implant component, as shown in FIGS. 2-4, for example, where protrusion 132 grips a liner 14 of an acetabular component 10.

Clamp 120 is also complemented by supplemental components to provide a secondary clamping effect. This includes a movable block 150, a block insert 160 disposable within and attachable to movable block 150, and a first locking member 140, as shown in FIGS. 2-4. Further, clamp 120 may optionally be further complemented by a second locking member 170 as shown in FIG. 2, as described further below.

In one variation that includes first locking member 140 and movable block 150, such components may be independent components that are assembled when extraction instrument 100 is used. In other variations, one or more of such components may be removably attached to a remainder of clamp 120 so that they are provided attached to the overall extraction instrument 100. Movable block 150 includes an inner part 152 and an outer part 154, the outer part including a projection 156. Outer part 154 may have an L-type shape in cross section with projection 156 extending inwardly into a closed-angle volume defined by outer part 154. Inner part 152 may extend from one side of outer part 154. Block insert 160 is shaped to be received within the volume defined by outer part 154 and includes a recess to complement projection 156. In some examples, the complementary projection and recess of movable block 150 and block insert 160 may be reversed compared to the arrangement shown in FIGS. 2-4. In other examples, outer part 154 and block insert 160 may include other features to facilitate engagement between such components. Block insert 160 may be attached to outer part 154 of movable block 150 through any attachment means, such as through the use of an adhesive. Block insert 160 may be made of softer material than movable block 150 to better interface with an implant surface to be gripped by extraction instrument 100. In some examples, block insert 160 may be made of polyphenylsulfone or silicone. In other examples, block insert 160 may be made of a metallic alloy of stainless steel. In examples where block insert 160 is made of a metallic alloy, block insert 160 and movable block 150 may be formed monolithically.

With continued reference to the supplemental components of clamp 120, first locking member 140 is positionable through an opening in base 122 and may be threadably engaged thereto. First locking member 140 may also include a drive 142 to allow for actuation of first locking member 140 with the use of a tool. First locking member 140 may be a fastener-type structure. Movable block 150 is sized to fit within and thereby be disposable within a volume between retention plate 138, base 122 and gripping arm 130. As will be described in greater detail in the method of using extraction instrument 100, a combination of first locking member 140 pressing against inner part 152 of block 150 and block 150 pressing against gripping member 130 may serve to clamp an object under block insert 160, such as lip 15 of acetabular cup 10. Such clamping action may be understood as a secondary grip or clamp, where protrusion 132 functions as a primary grip or clamp.

As mentioned above, in some variations of extraction instrument 100, clamp 120 may be complemented by first locking member 140, moveable block 150 and second locking member 170, as shown in combination in FIG. 2. While first locking member 140 may be tightened in a direction parallel to an elongate dimension of the shaft to secure block insert 160 against lip 15 of acetabular implant 10, second locking member 170 performs a similar function in a direction orthogonal to the engagement direction of first locking member 140 or, in other non-depicted examples, in another direction transverse to the engagement direction of first locking member 140. Specifically, second locking member includes a shaft 172 and a head 174, with the second locking member 170 being attached to clamp 120 such that shaft 172 passes through a cavity 153 in inner part 152 of movable block 150 and a free end of shaft 172 is movably engaged to gripping arm 130. Further, a second spring 178 or other biasing member may optionally be disposed in a space between movable block 150 and head 174 of second locking member 170. Second spring 178 may be biased in a compressed condition such that when no pulling force is applied to draw second locking member 170 away from movable block 150, second locking member 170 presses against movable block 150 and a tip region of shaft 174 may also press against gripping 130 to keep second locking member 170 in place. In other examples, second locking member 170 may secure movable block 150 to gripping arm 130 through complementary threads. In such instances, the second spring is not needed. Further details of how the aforementioned components are operable during use of the extraction instrument are provided elsewhere in the present disclosure in the description of the method of use.

In one non-depicted variation of extraction instrument 100 that does not include a second locking member transverse to a first locking member, a side of gripping arm 130 that faces retention plate 138 may include a channel sized to retain movable block 150 while allowing movable block 150 to move relative to base 122 of clamp 120. In such variation, movable block 150 may include a protruding ridge receivable in the channel. Such arrangement allows for movable block 150 to move toward and away from base 122 and also includes room in the connection so that movable block 150 may be moved toward and away from gripping arm 130, while simultaneously preventing movable block 150 from separating from a remainder of clamp 120 while not in use.

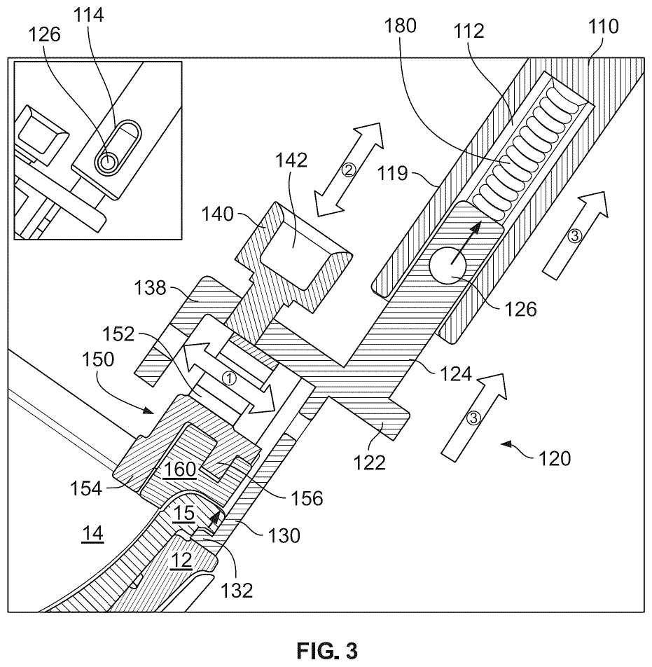

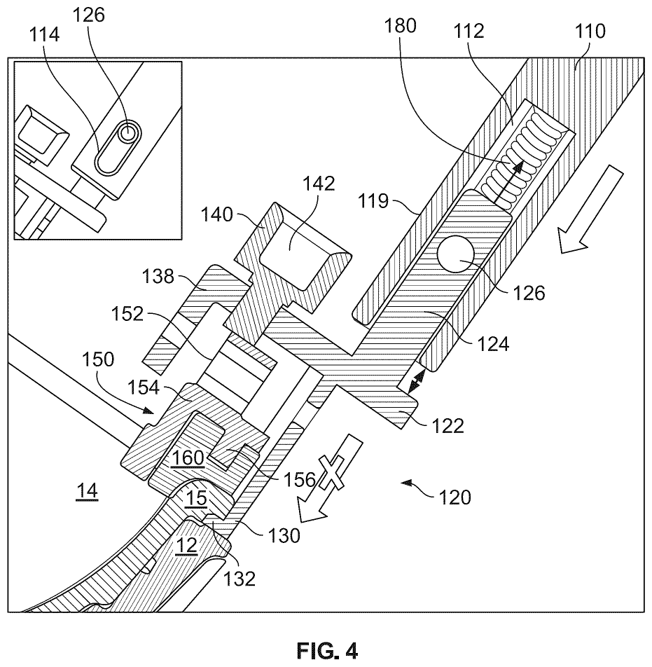

Extraction instrument 100 is advantageous in that when reciprocating action of shaft 110 relative to clamp 120 is activated through a mechanism such as a motor or other similar means located in the housing or otherwise remote from the clamping region, a relationship between a biased condition and stiffness of first spring 180 within cavity 112 and a reciprocating force generated by the mechanism is predetermined to control how forces are transferred from the shaft to the clamp. In this manner, when the reciprocating force is in a “pulling” phase and protrusion 132 is wedged between two implant components, shaft 110 pulls on clamp 120 as shown in FIG. 3, an end of slot 114 presses against pin 126, and there is a 1:1 transfer of energy from the shaft 110 to the clamp 120, providing a desired pull of one implant component from another. In some examples, the implant component pulled by the extraction instrument may be insert 14 held within acetabular shell 12. In contrast, undesirable forces are mitigated with extraction instrument 100. Specifically, with continued reference to the instrument having protrusion 132 wedged between two implant components, when the reciprocating force is in a “pushing” phase, as shown in FIG. 4, force from shaft 110 is dampened by first spring 180 that absorbs such force to greatly diminish such forces from being transferred to clamp 120. This “pull” and “push” extraction process is particularly advantageous in circumstances where the implant is in vivo in a patient and it is necessary to minimize the disturbance of the part of the implant that will remain in the patient.

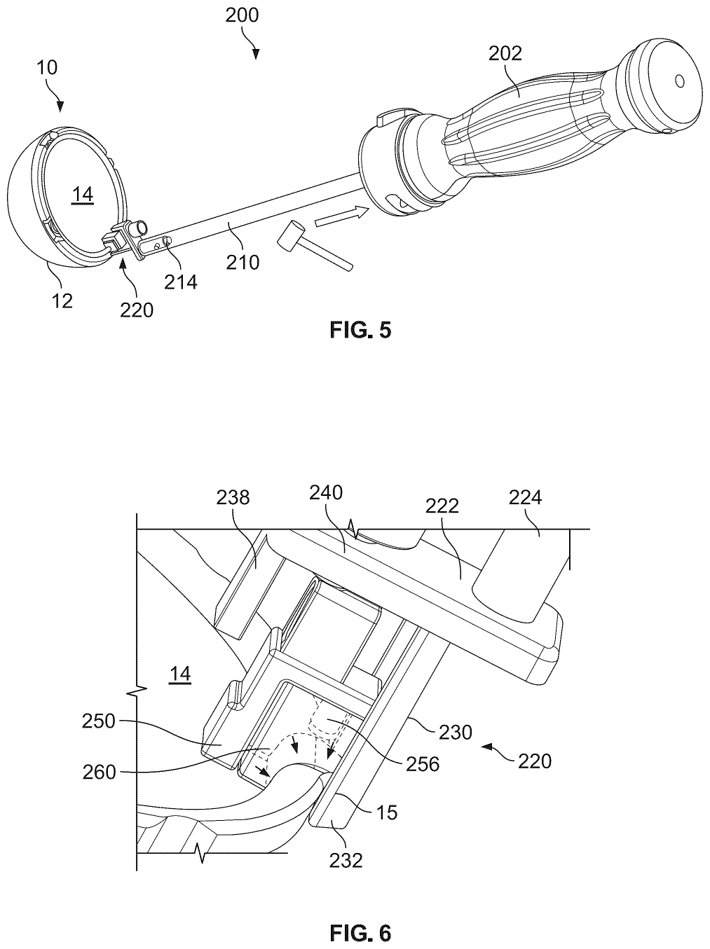

In one embodiment, an extraction instrument 200 is as shown in FIGS. 5-6. Unless otherwise indicated, like reference numerals refer to like elements of extraction instrument 100 shown in FIGS. 1-4, but within the 200-series of numerals. Extraction instrument 200 includes a handle 202, a shaft 210 and a clamp 220. Clamp 220 may include any combination of components as described for clamp 120 of instrument 100, and as shown in FIG. 6. In this manner, clamp 220 may provide multi-directional clamping on an implant component, such as clamping onto lip 15 of liner 12, with clamping along a direction parallel to shaft 210, and in one or more directions angled with respect to shaft 210. Extraction instrument 200 is a non-motorized device, and may be operated with a manually-applied impaction force on handle 202 to cause a reciprocating movement of shaft 210 relative to clamp 220, e.g., via a spring disposed within shaft 210.

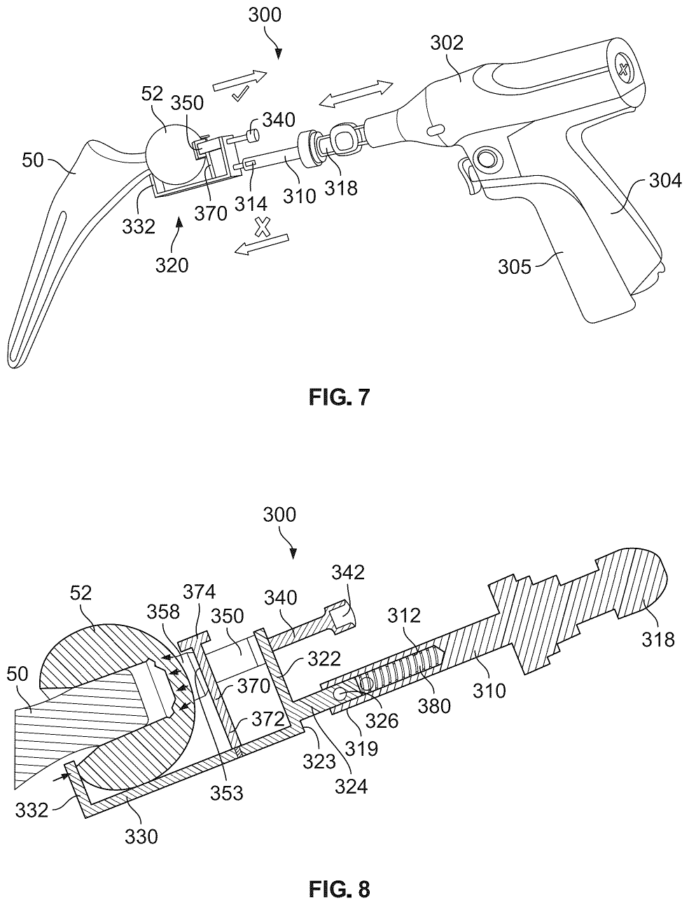

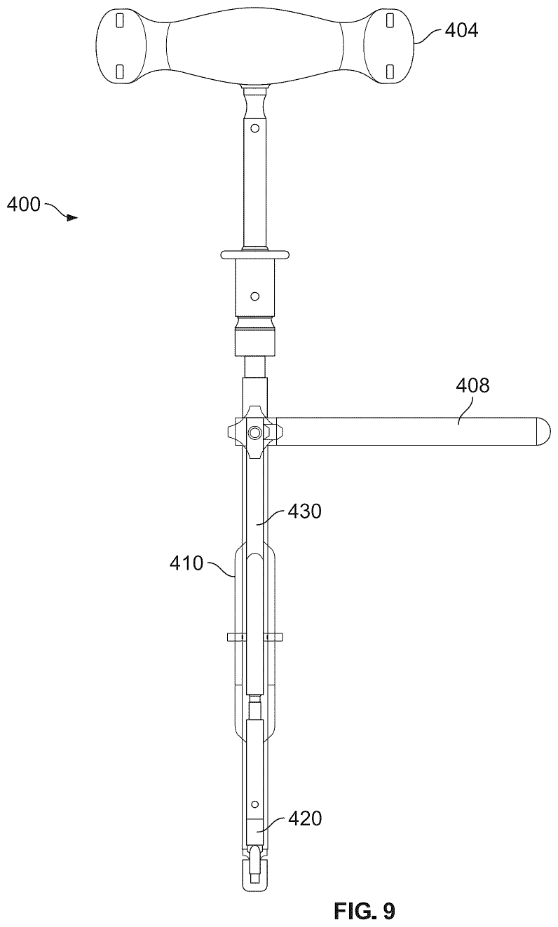

In one embodiment, an extraction instrument 300 is as shown in FIGS. 7-8. Unless otherwise indicated, like reference numerals refer to like elements of extraction instrument 100 shown in FIGS. 1-4, but within the 300-series of numerals. Extraction instrument 300 includes a housing 302, a shaft 310 and a clamp 320. Generally, extraction instrument 300 is very similar to extraction instrument 100, though adapted for use in clamping onto a femoral head 52 of a femoral implant and removing such femoral head 52 from a femoral stem 50 of the femoral implant when the femoral stem is in vivo in a patient. Extraction instrument 300 may operate via a mechanized reciprocating force transferred to shaft 310 from the mechanism and controlled via trigger 305, for example. Shaft 310 is removably engaged to housing 302, and clamp 320 is attached to shaft 310 in a similar manner to that described for the combination of shaft 110 and clamp 120. Clamp 320 includes a base 322 with a gripping arm 330 extending therefrom and receiving a first locking member 340 therein, as shown in FIG. 8. A second locking member 370 is optionally disposable through a cavity 353 of movable block 350 and into gripping arm 330 such that first locking member 340 may be adjustably engaged to movable block 350 to control whether movable block 350 is adjustable relative to gripping arm 330. Through loosening of first locking member 340 relative to movable block 350, movable block 350 may be adjusted to better seat a contact surface 358 of movable block 350 onto femoral head 52, thereby optimizing cooperation between the respective surfaces. Second locking member 370 may also be toggled between tight and loose configurations to control whether and to what extent movable block 350 is manipulable. In some examples, second locking member 370 may be threadably engageable with movable block 350 and gripping arm 330. In other examples, second locking member 370 may include a second spring (not shown) adjacent to a head of the second locking member to control locking of movable block 350 relative to gripping arm 330. An end of gripping arm 330 includes a protrusion 332. In use, protrusion 332 is positionable on an end of femoral head 52 opposite a pole of femoral head 52 that is engaged by contact surface 358 of movable block 350 to complete a grip on femoral head 52. Further details of a method of gripping and removal of implant components using extraction instrument 300 is provided elsewhere in the present disclosure.

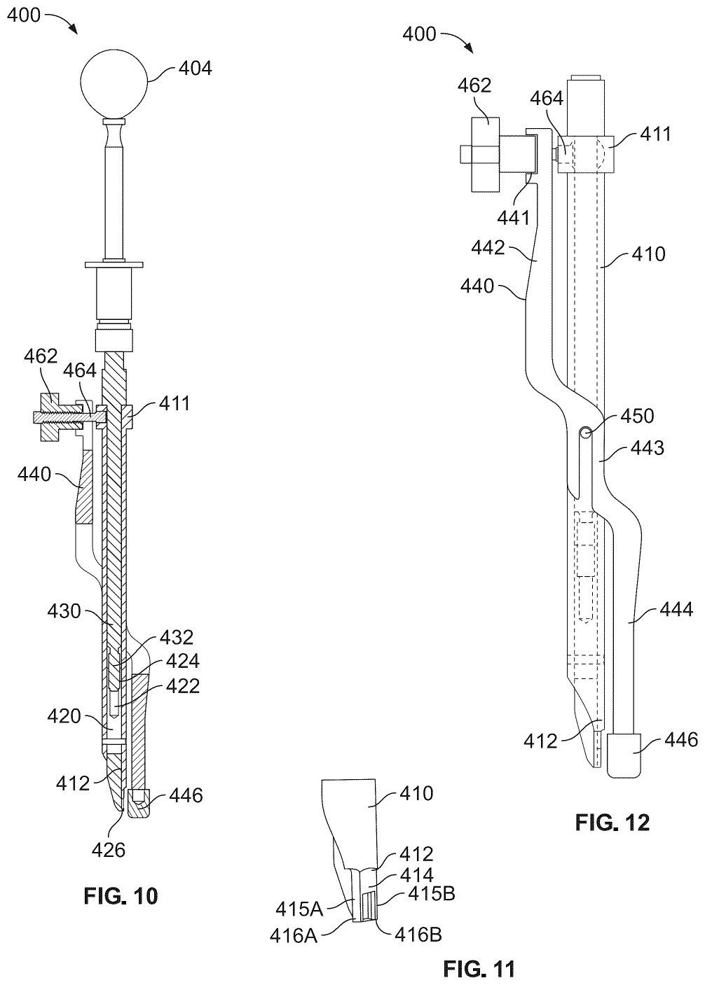

In one embodiment, an extraction instrument 400 is as shown in FIGS. 9-12. Extraction instrument 400 includes an engagement handle 404, a proximal inner shaft 430, a distal inner shaft 420 movably engaged to proximal inner shaft 430, an outer shaft 410 disposed over the inner shafts and a counter handle 408 extending from outer shaft 410. And, as shown in FIGS. 10 and 12, extraction instrument 400 also includes a clamp arm 440 attached to outer shaft 410 at a proximal end 411 of outer shaft 410 and at pin 450.

Turning initially to the shaft components, as shown in FIG. 10, proximal inner shaft 430 includes an end portion 432 remote from handle 404. End portion 432 includes a threaded surface 424. Distal inner shaft 420 includes a receiving cavity 422 defined by an inner surface that is threaded to complement threaded surface 424. In an assembled condition, distal inner shaft 420 is threadably engaged to proximal inner shaft 430 such that end portion 432 is received in receiving cavity 422. Portions of proximal inner shaft 430 and distal inner shaft 420, including where they are engaged, is enclosed within outer shaft 410. And, as described in greater detail elsewhere in the present disclosure, the manner of engagement between the inner shafts 420, 430 is such that rotation of proximal inner shaft 430 causes axial translation of distal inner shaft 420. Depending on the direction of rotation of proximal inner shaft 430, this results in distal inner shaft 420 moving towards a proximal end of the extraction instrument or moving away from the proximal end. Optionally, and included in extraction instrument 400 as depicted, distal inner shaft 420 may have a transverse cavity 428 as shown in FIG. 15B, and a pin 429 attached to outer shaft 410 may be disposed therethrough. In this way, an extent of axial translation of distal inner shaft 420 may be set to predetermined limits based on a length of transverse cavity 428 along the longitudinal direction of the shafts.

Outer shaft 410 has a length extending from proximal end 411 to distal end 412, and in an assembled state, is arranged such that a distal tip protrusion 426 of distal inner shaft 420 protrudes distally from distal end 412. Further, as shown in FIG. 11, Outer shaft 410 also includes an abutment portion 414 extending from distal end 412 along with first and second elongate prongs 415A, 415B extending from the abutment portion. First and second elongate prongs 415A-B are offset from a centerline of outer shaft 410 such that a position of distal tip protrusion 426 of distal inner shaft 420 is between prongs 415A-B along a range of translation of distal tip protrusion 426 relative to outer shaft 410.

As shown in FIG. 12, clamp arm 440 includes a proximal portion 442, a central portion 443 and a distal portion 444. Clamp arm 440 is shaped such that respective portions 442-444 are offset from each other. In this manner, in one plane through outer shaft 410 (i.e., through a length of outer shaft 410 and into the page in FIG. 12), proximal portion 442 may be on a first side of the plane, distal portion 444 may be on a second side opposite the first side, while central portion 443 may be generally in the plane. Central portion 443 includes a slot or another opening (e.g., closed or open) that receives a pin 450, pin 450 being attached to outer shaft 410. Distal portion 444 extends to a free end that includes an enlarged end portion 446. Such enlarged end portion 446 may be a softer material than a remainder of clamp arm 440. For example, enlarged end portion 446 may be made of polyphenylsulfone or silicone. Enlarged end portion 446 of the above and other examples may also be a foam. A proximal end of proximal portion 442 includes a receiving channel 441. Receiving channel 441 may include a through opening oriented in a direction transverse to a length of clamp arm 440 and may have a recessed surface region surrounding the through opening. As shown in FIGS. 10 and 12, such shape allows for an engagement shaft 464 to extend therethrough and for a knob 462 to be rotatably attached to engagement shaft 464. An end of engagement shaft 464 may be received within a side opening in outer shaft 410. Knob 462 and engagement shaft 464 may be operatively connected such that rotation of knob 462 causes knob 462 to advance or retract along engagement shaft 464, thereby controlling a rotational position of clamp arm 440 relative to pin 450. In turn, such movement causes enlarged end portion 446 to move closer to or further away from abutment portion 414 of outer shaft 410.

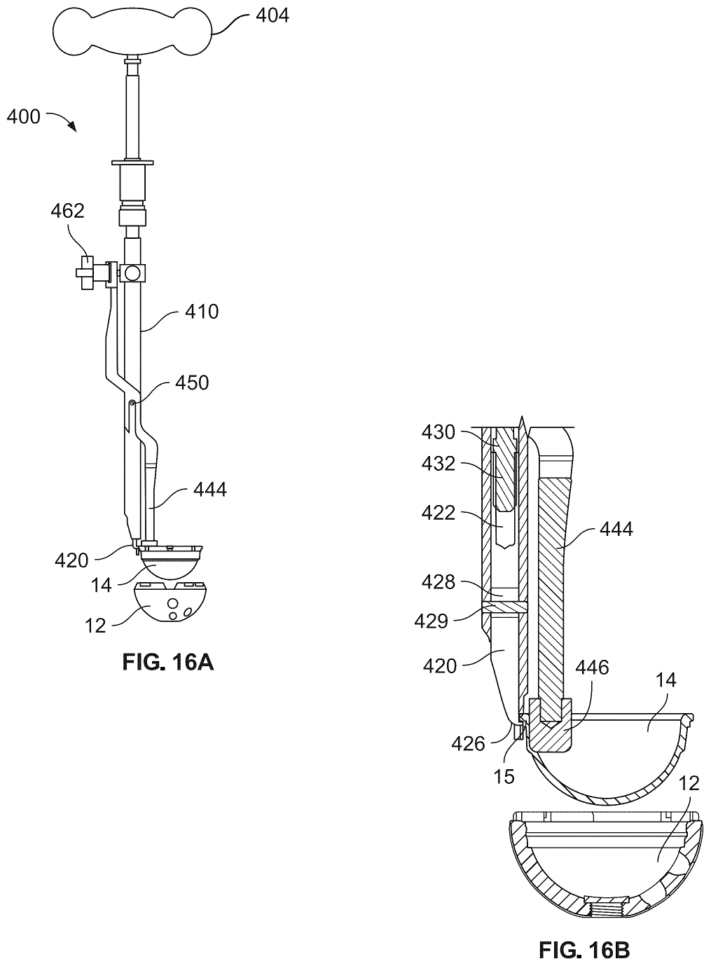

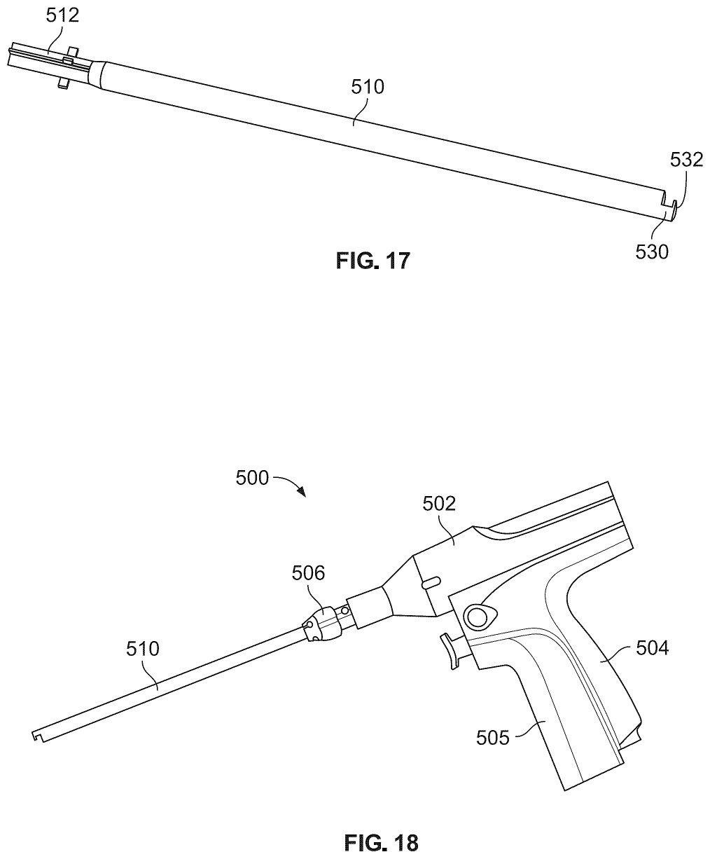

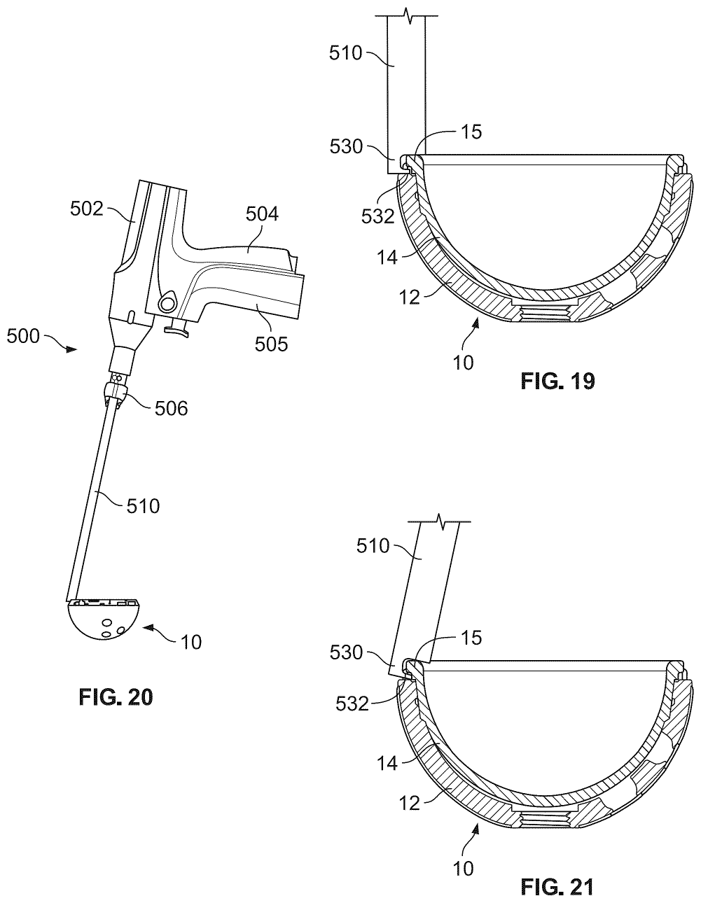

In one embodiment, an extraction instrument 500 is as shown in FIGS. 17-21. Unless otherwise indicated, like reference numerals refer to like elements of extraction instrument 100 shown in FIGS. 1-4, but within the 500-series of numerals. Extraction instrument 500 includes a housing 502 and an inner shaft 510 extending from housing 502. Inner shaft 510 may be attached to the housing via receipt of an interface structure 512 within receiving opening 506 of housing 502. Housing 502 includes a handle 504 with trigger 505 as described for extraction instrument 100. A mechanism such as a motor within the housing or operatively connected to the housing may be activated to cause reciprocating forces to be applied to shaft 510. Extending distally from a distal end of shaft 510 is a gripping arm 530. Gripping arm 530 includes a protrusion 532 that extends transversely to an elongate dimension of shaft 510. An end of shaft 510 and gripping arm 530 extending therefrom define a pocket, as shown in FIGS. 19 and 21, for example.

The extraction instrument may be made of various biocompatible materials. For instance, portions of the shaft and clamp may be made of titanium or stainless steel. Certain subcomponents may also be made of polymeric materials. Components that are designed for gripping and contacting implants may be made of porous materials, such as materials having a foam-type structure.

The extraction instrument may be varied in many ways. For example, a housing component of the extraction instrument in any of the contemplated embodiments may have a different shape from housing 102. Further, the mechanism for enabling the reciprocating function may be included on housing in a manner different from a pistol-grip trigger type structure. For example, a singular elongate housing with a button may form the housing component. In other examples, an arrangement of the various portions of the clamp may vary from that shown in the depicted embodiments. For instance, a location of the gripping arm and the receiving post relative to the base of the clamp may vary. Similarly, the protrusion at a free end of gripping arm may be oriented at any number of angles and have a size and shape that varies from that shown in the depicted embodiment, provided that it may serve to hold an implant component to be extracted from another implant component. Similarly, a shape of movable block 150 may also vary from that shown in the applicable depicted embodiments.

In another aspect, the present disclosure relates to an extraction instrument kit. In one embodiment, a kit may include two or more extraction instruments contemplated by the present disclosure, where at least two such extraction instruments are the same. In one embodiment, a kit may include two or more extraction instruments contemplated by the present disclosure, where at least two such extraction instruments are different. In some examples, the different extraction instruments may be the same embodiment of extraction instrument, but different sizes. In other examples, the different extraction instruments may be different embodiments of the extraction instrument. For instance, the kit may include extraction instrument 100 and extraction instrument 300. In any contemplated kit, the kit may include additional instruments, such as tools to complement extraction instrument, e.g., a mallet or a power supply for the powered operation of the instrument. Further, in any of the contemplated embodiments, a kit may include two or more user gripping components that may employ any number of mechanisms to control operation of a motor or other mechanism for the instrument. In other contemplated embodiments, a kit may include a single user gripping component, such as housing 102, and two or more sets of shaft and clamp combinations for attachment to the user gripping component. In this manner, even when a tool is desired for extraction of more than one type of implant component, such extraction may be accomplished with a single user gripping component by swapping out the clamp feature.

The kit may be varied in many ways. For example, the various combinations of elements of any kit contemplated herein may be included in a single package or distributed among multiple packages. In other examples, the kit contemplated herein may be accompanied by an instruction manual on how to perform one or more of the methods of using the contents of the kit.

In another aspect, the present disclosure relates to a method of extracting one implant component from another implant component. Such procedure may be performed in circumstances where a starting condition involves the implant being an existing implant previously implanted into a patient, and the treatment involves revision of such existing implant. Example procedures include, but are not limited to, removal of a liner from an acetabular shell of a hip implant and removal of a femoral head from a femoral stem of a hip implant. Other exemplary applications include shoulder revision, e.g., separation of glenoid implant components with taper locked junctions, and component separation for revision femoral stems, femoral sleeves and segmental limb salvage implants. The embodiments described below refer to specific implants for the sake of brevity and clarity, and should not be understood as limiting in any way.

In one embodiment, extraction instrument 100 is used to extract an implant. In one example, an implant component extracted with such instrument is an acetabular implant 10, as shown in FIGS. 1 and 3-4. Specifically, extraction instrument 100 is used to remove a liner 14 from a shell 12, where the shell is securely fixed to a bone of the patient, i.e., a pelvis. The purpose of the liner extraction is to facilitate replacement of the liner, while also avoiding or otherwise minimizing disruption of the shell 12, thereby preserving bone. One exemplary circumstance where such extraction may become necessary is where the implant is well integrated in a patient bone and a liner of the implant is to be replaced. Replacement of the liner may be required due to infection, instability, or aseptic loosening, among other reasons. Beginning with the acetabular implant exposed and the hip region being accessible to a user, a distal, or leading end of extraction instrument 100 is directed to a liner 14 of acetabular implant 10. Once such leading end is proximate the liner, protrusion 132 of extraction instrument 100 is positioned so that it is between a lip 15 of liner 14 and shell 12, as shown in FIGS. 2-4. Then, clamp 120 may be adjusted to fit block insert 160 against a side of lip 15 generally opposite protrusion 132. Specifically, first locking member 140, and if included, second locking member 170, are kept loose so that movable block 150 with block insert 160 therein may be pressed against lip 15. Once in the desired position, first locking member 140 is actuated, e.g., rotated to press first locking member 140 against movable block 150. Such actuation may be via a drive instrument inserted into drive 142. Where extraction instrument 100 includes second locking member 170, head 174 of second locking member 170 may be drawn back while movable block 150 is adjusted, and when movable block 150 is in the desired position, released to cause second locking member 170 to press against movable block 150 via spring 178. In such case, first locking member 140 may still apply a clamping force when tightened.

When clamp 120 is tightened to grip lip 15 of acetabular implant 10, the motor or other mechanism operative to effectuate reciprocating action in the shaft of extraction instrument 100 may be activated to commence the reciprocating action in shaft 110. Using housing 102, trigger 105 is squeezed to activate the mechanism. In some examples, the reciprocating motion of shaft 110 relative to clamp 120 continues commensurate with a duration that trigger 105 is held, stopping when trigger 105 is released. It is also contemplated that the mechanism may be configured in other predetermined ways to control such activation of the reciprocating motion. For instance, pressing trigger 105 once to activate, then pressing again to deactivate. While in operation with protrusion 132 held under lip 15, shaft 110 moves away from base 122 of clamp 120 in one phase of the reciprocating motion as shown in FIG. 3, and toward base 122 in another phase of the reciprocating motion as shown in FIG. 4. When moving away from base 122, shaft 110 pulls receiving post 124 of clamp via direct contact between a wall of slot 114 and pin 126 so that there is a 1:1 transfer of force between the components. As protrusion 132 is part of the same clamp structure as receiving post 124, protrusion 132 is pulled with shaft 110 during such phase of the shaft movement, causing lip 15 of liner 14 to be pried relative to shell 12. In contrast, while shaft 110 is moving toward base 122, a force in shaft 110 is absorbed, i.e. dampened by spring 180 so that forces from shaft 110 through gripping arm 130 and protrusion 132 are largely eliminated or otherwise minimized. This limits any undesirable pushing of shell 12 further into the bone from its already implanted position. An extent of dampening may be customized based on the characteristics of the spring used and the forces generated through the mechanism causing the reciprocating motion. The reciprocating motion repeats in rapid succession over many cycles until liner 14 is loosened sufficiently for removal from shell 12, while minimizing any disruption of the shell or the bone surrounding the shell.

The above described method is advantageous in that it removes the variability inherent in the use of manual tools to apply extraction forces and it reduces the time required and difficulty inherent in manual methods. Further, because the extraction instrument is operated with controlled distribution of forces while also dampening any forces that would otherwise push against the implant, the extraction instrument has a high degree of precision and mitigates the risk of injury that could otherwise result from the original implant component being left in place after the completion of the surgical procedure. Other advantages of the extraction instrument include the multi-directional clamping realized through the clamp. For instance, with extraction instrument 100, an implant component, such as lip 15 of liner 14, is clamped via protrusion 132 on an underside of lip 15, and via a surface of block insert 160 over a large area on a topside of lip 15, as shown in FIG. 2. Further, clamping forces of block insert 160 are applied in multiple directions, increasing the securement of the clamp. Such arrangement minimizes risk that the clamp may move relative to liner 14 during use.

In another embodiment, extraction instrument 200 is used to extract one implant component from another. A method of extraction with such instrument may be the same as described for extraction instrument 100 up to the time when extraction instrument 200 is securely clamped to implant 10. From such time, a manual tool, such as a mallet, may be used to apply extraction forces against the instrument. Such forces may be applied to a distally-facing end of handle 202, for example. Forces are applied repeatedly until liner 14 is loosened and otherwise removable from shell 12.

In another embodiment, extraction instrument 300 is used to extract one implant component from another. Unless otherwise noted, a method of extraction with extraction instrument 300 may be as described for extraction instrument 100. Though generally the same, a process of clamping instrument 300 to a femoral head 52 may vary slightly based on a shape of such femoral head. Specifically, after protrusion 332 is positioned under femoral head 52, movable block 350 is adjusted so that a concave contact surface 358 sits flush on femoral head 52. Once properly seated, first locking member 340 is tightened to secure a position of movable block 350 relative to a remainder of clamp 320. Second locking member 370, where included, may then be tightened. Such tightening may be manual or via a spring as described for extraction instrument 100. Once clamp 320 is secured relative to femoral head 52, a mechanism, e.g., motor (not shown) of extraction instrument 300 is activated to cause pulling forces to be applied to femoral head 52 relative to femoral stem 50 via reciprocating action of shaft 310 relative to receiving post 324. During the performance of the aforementioned step, femoral stem 50 is anchored within a femur of a patient, and the reciprocating action of shaft 310 causes femoral head 52 to be removed from femoral stem 50.

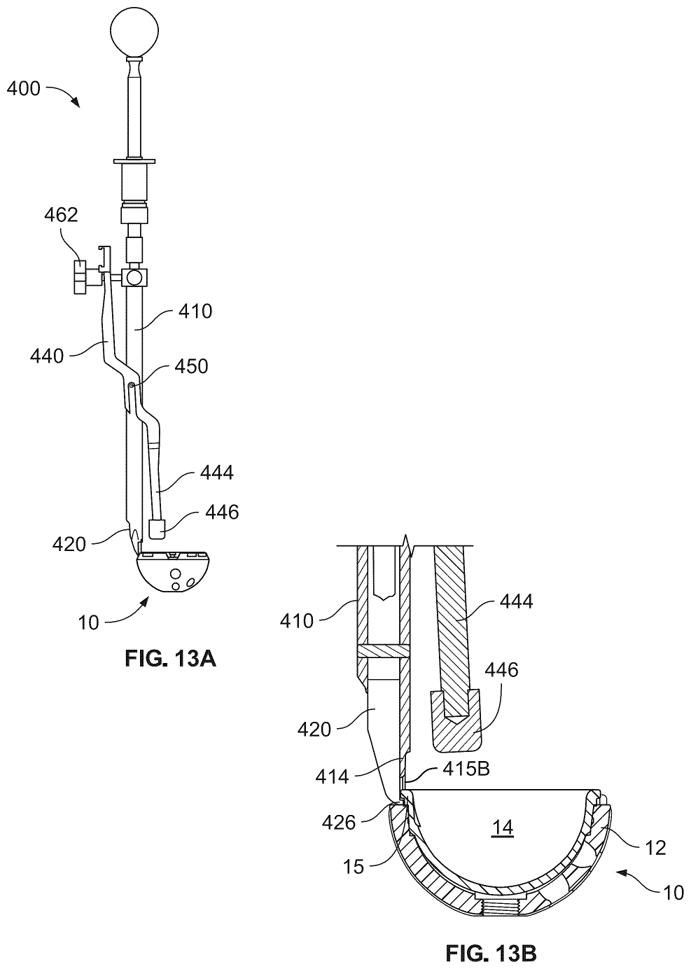

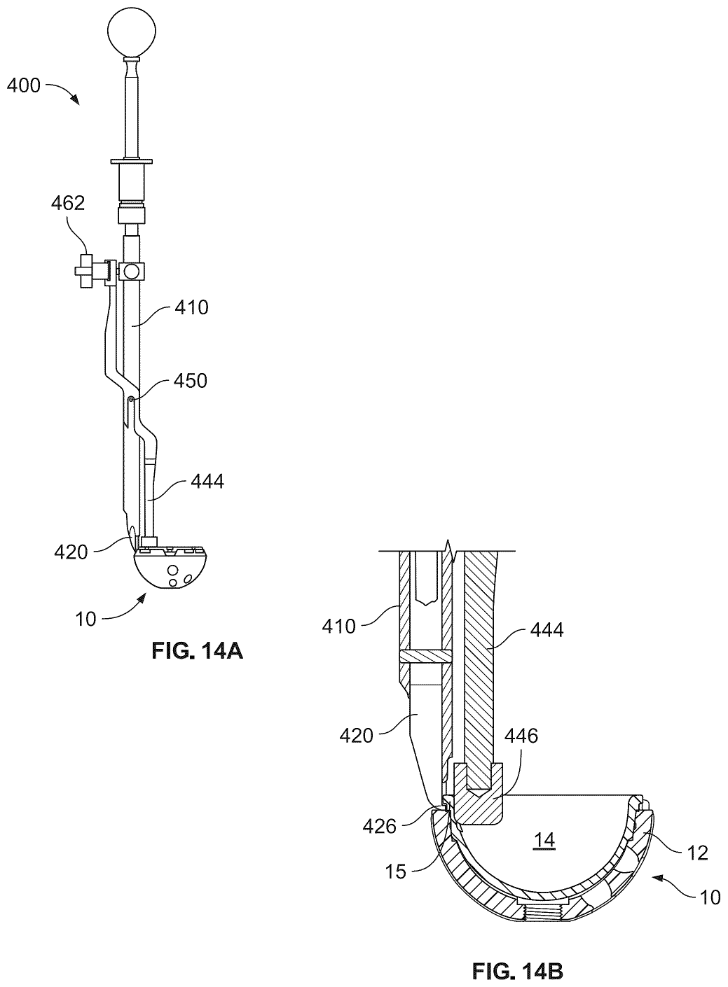

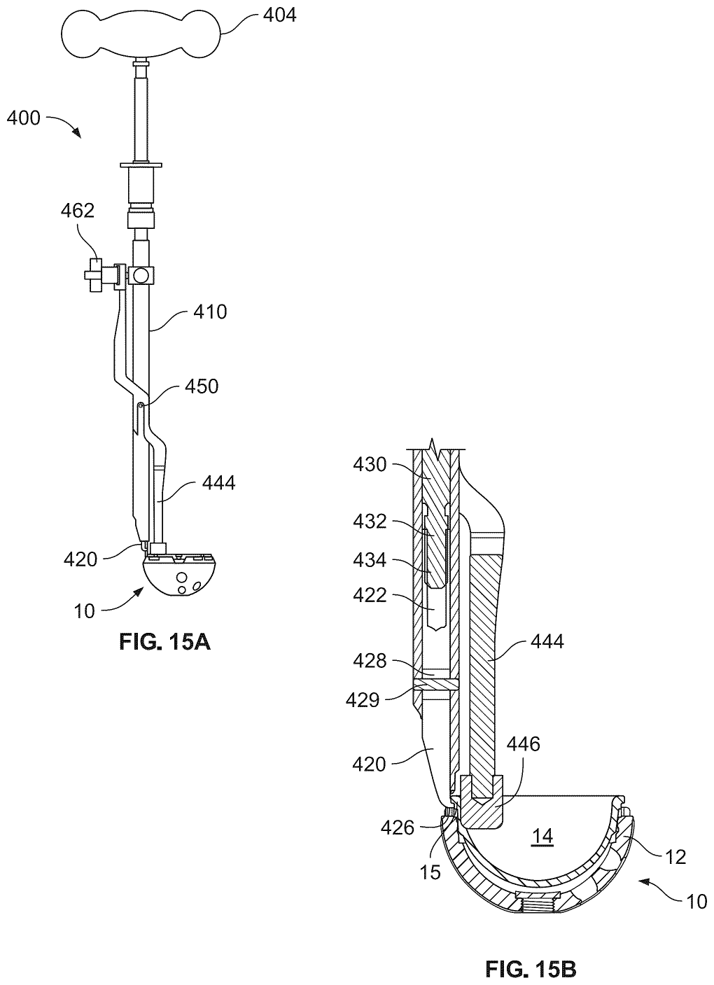

In yet another embodiment, extraction instrument 400 is used to extract one implant component from another. A method of extraction with such instrument is shown in FIGS. 13A-16B, where extraction instrument 400 is used to extract a liner 14 from a shell 12 of an acetabular implant 10, the method being performed with the shell securely fixed to a bone of a patient. Beginning with the acetabular implant exposed and accessible to a user, a distal, or leading end of extraction instrument 100 is directed to liner 14. Once such leading end is proximate the liner, distal tip protrusion 426 of extraction instrument 400 is positioned so that it is between a lip 15 of liner 14 and shell 12, as shown in FIGS. 13A-B. Further, in this position, first and second elongate prongs 415A, 415B press against or otherwise abut a rim of shell 12. Then, clamp arm 440, which may in some cases initially be positioned such that engaged end portion 446 is remote from tip protrusion 426, may be lowered so that an inner end of a slot of clamp arm 440 rests on pin 450, as seen in FIGS. 12 and 14A. Knob 462 is then rotated, e.g., clockwise, to bring knob 462 closer to outer shaft 410, thereby pushing proximal portion 442 and rotating clamp arm 440 about pin 450. This causes elongated end portion 446 to move toward lip 15 as shown in FIGS. 14A-B. Knob is rotated until liner 14 is tightly gripped from opposite sides by tip protrusion 426 of distal inner shaft 420 and enlarged end portion 446, respectively.

Once clamping is completed, handle 404 is rotated, e.g., clockwise, to rotate proximal inner shaft 430. As proximal inner shaft 430 rotates, distal inner shaft 420 axially translates in a proximal direction so that distal tip protrusion 426 withdraws toward a lumen of outer shaft 410 and away from ends 416A-B of respective first and second elongate prongs 415A-B. This process is illustrated by a comparison of a relative position of distal tip protrusion 426 in FIGS. 14A-B and 15A-B. During the actuation of the inner shaft, ends 416A-B of elongate prongs 415A-B that extend from outer shaft 410 remain pressed against or abut the rim of shell 12 while distal tip protrusion 426 is withdrawn toward a lumen of outer shaft 410, thereby prying liner 14 from shell 12. Rotation of handle 404 continues until distal tip protrusion 426 pries liner 14 from shell 12. Further, liner 14 remains clamped by distal tip protrusion 426 and elongated end portion 446 during the entire process such that when liner 14 is separated from shell 12, as shown in FIGS. 16A-B, liner 14 remains held by extraction instrument 400 for safe removal from the patient.

In another embodiment, extraction instrument 500 is used to extract one implant component from another. A method of extraction with such instrument is shown in FIGS. 19-21. In one example, extraction instrument 500 is used to extract a liner 14 from a shell 12 of an acetabular implant 10 in place within a patient. Beginning with the acetabular implant exposed and accessible to a user, a distal, or leading end of extraction instrument 500 is directed to liner 14. Once such leading end is proximate the liner, protrusion 532 of extraction instrument 500 is positioned so that it is between a lip 15 of liner 14 and shell 12, as shown in FIG. 19. At this juncture, an orientation of shaft 510 may be adjusted as desired by the user. In one example, and as shown in FIGS. 20-21, shaft 510 may be rotated over a cavity of implant 10. Once extraction instrument 500 is in position, a mechanism such as a motor may be activated through trigger 505 to cause shaft 510 to undergo a reciprocating motion. After a sufficient number of cycles, liner 14 is loosened and then removable from shell 12.

It should be appreciated that any of the instruments, implants and methods disclosed herein may be used in conjunction with robotic technology. For example, any of the extraction instruments described herein may be used with robotic surgical systems to extract an implant component from within a patient. An extraction instrument may be controlled by a robotic system or a robotic arm to automatically effectuate one or more of positioning of the instrument on the implant, clamping of the instrument on the implant, and removal of the implant from the patient. Further, any or all of the steps described in the contemplated methods of extracting an implant may be performed using a robotic system.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

1. An implant extraction instrument comprising:
a shaft;
a first gripping member movable relative to the shaft, the first gripping member including an elongate portion and a protrusion extending from the elongate portion in a direction transverse to a central longitudinal axis of the shaft; and
a second gripping member connected to the first gripping member, a position of the second gripping member being adjustable relative to the first gripping member,
wherein when the protrusion and the second gripping member are positioned to grip a first implant component therebetween, a position of the shaft relative to the first gripping member is controllable to cause the first implant component to be pulled away from a second implant component to which the first implant component is attached.

2. The implant extraction instrument of claim 1, wherein the first gripping member is translatable relative to the shaft.

3. The implant extraction instrument of claim 2, wherein a rotational position of the first gripping member relative to the shaft is fixed.

4. The implant extraction instrument of claim 2, wherein the shaft is an outer shaft and the first gripping member is a clamp, and the outer shaft and the clamp are operatively connected through a biasing member.

5. The implant extraction instrument of claim 2, wherein the second gripping member includes a locking member to set a position of an implant-interfacing surface of the second gripping member relative to the first gripping member.

6. The implant extraction instrument of claim 1, further comprising a housing connected to the shaft, the housing including a motor operatively connected to the shaft.

7. The implant extraction instrument of claim 1, wherein the first gripping member is disposed within a cannulation of the shaft and the first gripping member includes a proximal shaft and a distal shaft configured such that rotation of the proximal shaft causes the distal shaft to translate relative to the proximal shaft.

8. The implant extraction instrument of claim 1, wherein the second gripping member is pivotably attached to the first gripping member.

9. An implant extraction instrument comprising:
a handle member including a shaft extending from a proximal end to a distal end;
a grip member movably attached to the shaft proximate the distal end of the shaft, the grip member including a gripping arm configured to hold an implant;
a biasing member with a first end connected to the shaft and a second end connected to the grip member,
wherein when the grip member is attached to an implant in a patient, the shaft is movable relative to the grip member such that when the shaft is pushed toward the grip member, at least a portion of a force in the shaft that is associated with the pushing is absorbed by the biasing member, and when the shaft is pulled away from the grip member, a force in the shaft that is associated with the pulling is transferred to the grip member.

10. The implant extraction instrument of claim 9, wherein the shaft further comprises a slot and the grip member further comprises a pin disposed through the slot, a position of the pin within the slot changing with movement of the grip member relative to the shaft.

11. The implant extraction instrument of claim 9, wherein a portion of the grip member is disposed within the shaft and the biasing member is entirely disposed within the shaft.

12. The implant extraction instrument of claim 9, wherein the gripping arm has a free end with a protrusion extending transversely relative to the gripping arm, the gripping arm being configured to hold the implant.

13. The implant extraction instrument of claim 12, wherein the protrusion of the gripping arm is offset from a central longitudinal axis of the shaft.

14. The implant extraction instrument of claim 9, further comprising a supplemental grip member movably attached to the grip member, the supplemental grip member being shaped to clamp the implant in a direction different from that of the gripping arm against the implant.

15. An implant extraction instrument comprising:
an outer shaft having an elongate dimension extending along a central longitudinal axis;
an inner shaft movably disposed in the outer shaft, the inner shaft including a proximal portion and a distal portion translatable relative to the proximal portion, the distal portion including a tip protrusion extending transversely relative to the central longitudinal axis; and
a clamp arm rotatably attached to the outer shaft, the clamp arm including an end portion moveable relative to the tip protrusion,
wherein the tip protrusion and the end portion are configured to be movable to grip an object therebetween.

16. The implant extraction instrument of claim 15, wherein the outer shaft further comprises an elongate prong extending from a distal end of the outer shaft, the elongate prong extending distally of a lumen of the outer shaft such that an end of the distal portion of the inner shaft is positionable adjacent to the elongate prong outside of the lumen.

17. The implant extraction instrument of claim 15, wherein the proximal portion of the inner shaft is rotatable to cause the distal portion of the inner shaft to translate relative to the proximal portion, thereby translating tip protrusion relative to a distal end of the outer shaft.

18. The implant extraction instrument of claim 15, wherein a rotational position of the distal portion relative to the outer shaft is fixed.

19. The implant extraction instrument of claim 15, wherein the clamp arm is pivotable relative to the outer shaft.

20. The implant extraction instrument of claim 19, wherein a pivot axis of the clamp arm relative to the outer shaft is at a first location on the outer shaft and a locking member of the clamp arm configured to lock a rotational position of the clamp arm relative to the outer shaft is at a second location on the outer shaft spaced apart from the first location.
